(19) United States
(12) Patent Application Publication
(10) Pub. No.: US 2022/0243241 A1
(43) Pub. Date: Aug. 4, 2022

(54) Co-Culture of Myxobacteria and Pseudomonas for Enhanced Production of Biosurfactants and Other Metabolites

(71) Applicant: Locus IP Company, LLC, Solon, OH (US)

(72) Inventors: Sean FARMER, Ft. Lauderdale, FL (US); Ken ALIBEK, Solon, OH (US); Yajie CHEN, Solon, OH (US)

(21) Appl. No.: 17/620,324

(22) PCT Filed: Jun. 15, 2020
(86) PCT No.: PCT/US2020/037766

§ 371 Date: Dec. 17, 2021

Related U.S. Application Data
(60) Provisional application No. 62/862,180, filed on Jun. 17, 2019.

(51) Int. Cl. C12P 19/44; C12P 39/00; C12N 1/20; C12P 13/02; C12P 23/00

(52) U.S. Cl. CPC C12P 19/44; C12P 39/00; C12P 23/00; C12P 13/02; C12N 1/20

(57) ABSTRACT

Methods are provided for enhanced production of microbial biosurfactants, the methods comprising co-cultivating Myxococcus xanthus and Pseudomonas chlororaphis. In certain embodiments, the biosurfactants are rhamnolipids or rhamnolipids-like glycolipids. In certain embodiments, other microbial growth by-products are produced, such as organic hydrocarbons including terpenes and/or terpenoids. Microbe-based products produced according to the subject methods are also provided, as well as their uses in, for example, agriculture, oil and gas recovery, and health care.

### CROSS-REFERENCE TO RELATED APPLICATION

This application claims priority to U.S. Provisional Patent Application No. 62/862,180, filed Jun. 17, 2019, which is incorporated herein by reference in its entirety.

### BACKGROUND OF THE INVENTION

Cultivation of microorganisms such as bacteria, yeasts and fungi is important for the production of a wide variety of useful bio-preparations. Microorganisms play crucial roles in, for example, food industries, pharmaceuticals, agriculture, oil and gas recovery, mining, environmental remediation, and waste management; however, one of the factors restricting commercialization of microbe-based products has been the cost per propagule density, as it is particularly expensive and unfeasible to produce microbes and their growth by-products on a large scale.

Interest in microbial surfactants, i.e., biosurfactants, in particular, has been steadily increasing in recent years due to structural diversity, environmental-friendliness, selectivity, performance under extreme conditions, and potential “green” applications in various industries.

Biosurfactants are a structurally diverse group of surface-active substances produced by microorganisms. All biosurfactants are amphiphiles comprising two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces.

Biosurfactants can also reduce the interfacial tension between water and oil and, therefore, lower the hydrostatic pressure required to move entrapped liquid to overcome the capillary effect. By accumulating at interfaces, biosurfactants can reduce interfacial tension, leading to the formation of aggregated micellar structures in solution. The formation of micelles provides a physical mechanism to mobilize, for example, oil in a moving aqueous phase, and can have powerful emulsifying and demulsifying properties. The ability of biosurfactants to form pores and destabilize biological membranes also permits their use as antibacterial, antifungal, and hemolytic agents to, for example, control pests, inhibit microbial adhesion to a variety of surfaces, and/or prevent the formation of biofilms. Furthermore, biosurfactants are biodegradable, have low toxicity, and can be economically produced using renewable resources.

Combined with the characteristics of low toxicity and biodegradability, biosurfactants can be useful in a variety of settings and industries. Most biosurfactant-producing organisms produce biosurfactants in response to the presence of a hydrocarbon source in the growing media. Other media components, such as concentration of minerals and pH, can also affect biosurfactant production significantly.

Microbial biosurfactants are produced by a variety of microorganisms such as bacteria, fungi, and yeasts, including, for example, Starmerella spp. (e.g., S. bombicola), Pseudomonas spp. (e.g., P. aeruginosa, P. putida, P. florescens, P. fragi, P. syringae); Flavobacterium spp.; Bacillus spp. (e.g., B. subtilis, B. amyloliquefaciens, B. pumillus, B. cereus, B. licheniformis); Wickerhamomyces spp. (e.g., W. anomalus), Candida spp. (e.g., C. albicans, C. rugosa, C. tropicalis, C. lipolytica, C. torulopsis); Saccharomyces (e.g., S. cerevisiae); Pseudozyma spp. (e.g., P. aphidis); Rhodococcus spp. (e.g., R. erythropolis); Ustilago spp. (e.g., U. maydis); Arthrobacter spp.; Campylobacter spp.; Cornybacterium spp.; as well as others.

There are multiple types of biosurfactants, which include low molecular weight glycolipids, lipopeptides, flavolipids and phospholipids, and high molecular weight polymers such as lipoproteins, lipopolysaccharide-protein complexes, and polysaccharide-protein-fatty acid complexes. The hydrocarbon chain of a fatty acid acts as a common lipophilic moiety of a biosurfactant molecule, whereas the hydrophilic part can be formed by, for example, esters, alcohols, carboxylates, amino acids, peptides and/or carbohydrates.

One important type of biosurfactant is rhamnolipids (RLP). RLP are glycolipids comprising a rhamnose moiety and a 3-(hydroxyalkanoyloxy)alkanoic acid fatty acid tail. Two main classes of rhamnolipids exist, mono-rhamnolipids and di-rhamnolipids, which have one or two rhamnose groups, respectively. The length and degree of branching in the fatty acid tail can also vary between RLP molecules.

Most commonly, RLP are produced using the bacterium Pseudomonas aeruginosa; however, P. aeruginosa is an opportunistic pathogen that can be fatal in humans, particularly those with compromised immune systems. The pathogenic nature of P. aeruginosa thus limits the application of RLP, in part because of the risks it poses to the people who work with it.

Additional microbial growth by-products that can be useful in a variety of industries include, for example, biopolymers, acids, enzymes, antibiotics, antivirals, antifungals, and solvents; however, one limiting factor in commercialization of microbe-based products has been the difficulty in developing safe, large scale operations for cultivating efficacious microbial products.

Two principle forms of microbe cultivation exist for growing microbes and producing their growth by-products: submerged (liquid fermentation) and surface cultivation (solid-state fermentation (SSF)). SSF utilizes solid substrates, such as bran, bagasse, and paper pulp, for culturing microorganisms. One advantage to this method is that nutrient-rich waste materials can be easily recycled as substrates. Additionally, the substrates are utilized very slowly and steadily, so the same substrate can be used for long fermentation periods.

Submerged fermentation utilizes free flowing liquid substrates, such as molasses and nutrient broth. Bioactive compounds can be secreted by the growing microbes into the flowing liquid.

Microbes and their growth by-products have the potential to play highly beneficial roles in a variety of industries, including petroleum, agriculture, and cosmetics; however, safer and more efficient methods are needed for producing the large quantities of microbe-based products, such as rhamnolipids and RLP-like substances, that are required for such applications.

### BRIEF SUMMARY OF THE INVENTION

The subject invention provides improved methods of producing microbial biosurfactants and other useful microbial metabolites. Advantageously, the methods and microbe-based products of the subject invention are environmentally-friendly, operational-friendly and cost-effective.

In preferred embodiments, the subject invention provides methods of producing one or more microbial growth by-products, the methods comprising co-cultivating a myxobacterium and a Pseudomonas spp. bacterium. Advantageously, in certain embodiments, the total cell biomass and/or the total production of the one or more growth by-products achieved when using the subject methods is greater than when pure cultures of the individual microbes are cultivated.

In a specific embodiment, the myxobacterium is Myxococcus xanthus and the Pseudomonas is P. chlororaphis. In preferred embodiments, the Pseudomonas is a strain that is not pathogenic to humans, such as, e.g., P. chlororaphis strain 111 (UCM B-111) or P. chlororaphis strain 306 (UCM B-306). In certain embodiments, more than one strain of Pseudomonas can be utilized.

In one embodiment, the microorganisms are co-cultivated using cultivation processes ranging from small to large scale. These cultivation processes can include, but are not limited to, submerged cultivation/fermentation, solid state fermentation (SSF), and hybrids, modifications and/or combinations thereof In some embodiments, the cultivation process is a fed-batch process.

In one embodiment, co-cultivation is carried out using submerged fermentation. In one embodiment a hybrid of SSF and submerged fermentation is used, wherein a particulate anchoring carrier is suspended in the liquid culture medium to serve as a site for cell attachment and biofilm formation. This is particularly useful for the growth of myxobacteria, which can exhibit enhanced growth on a solid surface.

The liquid growth medium can comprise sources of, for example, carbon, nitrogen, proteins, vitamins and/or minerals. In certain embodiments, the nutrient medium is customized for production of a high concentration of one or more specific microbial growth by-products. In one embodiment, the liquid nutrient medium comprises a foam preventer, such as, for example, canola oil.

In some embodiments, the particulate anchoring carrier is suspended in the liquid culture medium prior to, concurrently with, or after the liquid culture medium is inoculated with the first and/or second microorganisms.

In one embodiment, the anchoring carrier can be any sterilized material suitable for serving as a nucleation site for bacterial attachment and growth. In some embodiments, the material comprises a plurality of individual fine particles, e.g., grains, which are about 0.1 μm to about 5 mm in diameter. Bacteria will attach to the particles and accumulate thereon, producing bacterial-carrier masses.

The anchoring carrier can be inert, or it can carry and/or comprise additional nutrients and/or microbial inoculant. In certain embodiments, the anchoring carrier can be porous. The anchoring carrier can comprise synthetic materials and/or naturally-derived materials.

In one embodiment, the anchoring carrier comprises balls made of, for example, glass, a polymer (e.g., polylactic acid (PLA)), agar, or gelatin. In one embodiment, the anchoring carrier can be pieces of, for example, a chopped sponge or loofa. In one embodiment, the anchoring carrier can comprise foodstuff, such as seeds, nuts, beans or even pieces of chopped fruit, such as bananas.

In preferred embodiments, the anchoring carrier comprises fine grains of cellulose and/or corn flour.

Advantageously, the use of the anchoring carrier provides for increased production of bacterial biomass due to, for example, the increased surface area on which the bacteria can attach and accumulate. Additionally, the accumulation of bacterial biomass can lead to increases in the production of beneficial growth by-products, such as biosurfactants and other metabolites.

In one embodiment, bacteria grow in the form of a biofilm on the particulate anchoring carrier. In one embodiment, some bacteria grow in the liquid culture medium and some bacteria grow on the particulate anchoring carrier.

In some embodiments, the cultivation method utilizes fed-batch cultivation. The fermentation reactor can be fed with, for example, canola oil (or another anti-foam solution), carbon sources (e.g., glycerol), pH adjusters, and/or other additional nutrient sources as needed. “Feeding” of the fermentation reactor can occur, for example, at 24 hours, at 48 hours, or multiple times, for example, every 24 to 48 hours.

According to the subject methods, the first and second microorganisms can be incubated in the fermentation system for a time period sufficient to achieve a desired effect, e.g., production of a desired amount of cell biomass or a desired amount of one or more microbial growth by-products. In some embodiments, fermentation occurs for 24 hours to 5 days or longer, at a temperature of 20 to 30° C.

In preferred embodiments, the methods of the subject invention can be used to produce one or more microbial growth by-products. In certain embodiments, the growth by-products comprise one or more biosurfactants.

Biosurfactants according to the subject invention can include, for example, glycolipids, lipopeptides, flavolipids, phospholipids, high-molecular-weight polymers, fatty acid esters, fatty acid ethers, lipoproteins, lipopolysaccharide-protein complexes, and/or polysaccharide-protein-fatty acid complexes.

In specific embodiments, the methods can be used to produce glycolipid biosurfactants, such as, for example, rhamnolipids (RLP), trehalose lipids, mannosylerythritol lipids (MEL), cellobiose lipids and/or sophorolipids (SLP). In certain embodiments, the methods can be used to produce from 5 to 30 g/L of glycolipids. In preferred embodiments, the glycolipids are rhamnolipids and/or RLP-like glycolipids.

In certain embodiments, more than one type of biosurfactant is produced during co-cultivation, for example, other glycolipids and/or flavolipids.

In some embodiments, the one or more growth by-products can also include other metabolites, for example, enzymes, biopolymers, acids, solvents, gases, proteins, peptides, amino acids, alcohols, hormones, lipids, carbohydrates, antibiotics, pigments, and other bioactive compounds. In a specific embodiment, the other metabolites are terpenes and/or terpenoids, such as, for example, carotenoids.

Advantageously, in certain embodiments, the methods of the subject invention can result in the production of biosurfactants, terpenes and/or terpenoids, and/or other growth by-products at greater concentrations than when pure cultures of the individual microbes are cultivated.

In certain embodiments, the subject invention provides microbe-based products produced according to the subject methods, as well as their uses in, for example, improved oil production, bioremediation and mining; waste disposal and treatment; promoting plant health and productivity; and reclaiming and/or restoring the health of soils.

The microbe-based products can comprise the entire culture produced according to the subject methods, including the first and/or the second microorganisms and/or their growth by-products, as well as residual growth medium, particulate anchoring carrier and/or nutrients.

The microorganisms can be live, viable or in an inactive form. They can be in the form of a biofilm, vegetative cells, spores, and/or a combination thereof. In certain embodiments, no microbes are present, wherein the composition comprises microbial growth by-products, e.g., biosurfactants, which have been extracted from the culture and, optionally, purified.

### DETAILED DESCRIPTION

The subject invention provides methods of producing microorganisms and their growth by-products. Advantageously, the microbe-based products and methods of the subject invention are environmentally-friendly, operational-friendly and cost-effective.

In preferred embodiments, the subject invention provides methods for enhanced production of one or more microbial growth by-products, the methods comprising co-cultivating a myxobacterium and a strain of Pseudomonas spp. In a specific embodiment, the growth by-products include biosurfactants, such as rhamnolipids and/or flavolipids.

The growth by-products can also include other metabolites, for example, enzymes, biopolymers, acids, solvents, gases, proteins, peptides, amino acids, alcohols, hormones, lipids, carbohydrates, antibiotics, and other organic and/or bioactive compounds. In a specific embodiment, the other metabolites are terpenes and/or terpenoids, such as, for example, carotenoids.

Advantageously, in certain embodiments, the total cell biomass and/or the total production of the one or more growth by-products achieved according to the subject methods can be greater than when pure cultures of the individual microbes are cultivated on their own.

### Selected Definitions

As used herein, a “biofilm” is a complex aggregate of microorganisms, such as bacteria, wherein the cells adhere to each other and/or to a surface using an extracellular polysaccharide matrix. The cells in biofilms are physiologically distinct from planktonic cells of the same organism, which are single cells that can float or swim in liquid medium.

As used herein, “co-cultivation” means cultivation of more than one microorganism in a single fermentation system. In some instances, the microorganisms interact with one another, either antagonistically or symbiotically, resulting in a desired effect, e.g., a desired amount of cell biomass growth or a desired amount of metabolite production. In one embodiment, this antagonistic or symbiotic relationship can result in an enhanced effect, for example, the desired effect can be magnified when compared to what results from cultivating only one of the microorganisms on its own. In an exemplary embodiment, one microorganism, e.g., a Myxococcus sp., can serve as a stimulator for the production of biosurfactants or other metabolites by the other microorganism, e.g., a Pseudomonas sp.

As used herein, “enhancing” refers to improving and/or increasing.

As used herein, “fermentation” refers to cultivation or growth of cells under controlled conditions. The growth could be aerobic or anaerobic.

As used herein, an “isolated” or “purified” nucleic acid molecule, polynucleotide, polypeptide, protein, organic compound such as a small molecule (e.g., those described below), or other compound is substantially free of other compounds, such as cellular material, genes or gene sequences, and/or amino acids or amino acid sequences, with which it is associated in nature. A purified or isolated microbial strain is removed from the environment in which it exists in nature and/or in which it was cultivated. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is, preferably, at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state or in spore form, or a mixture of both. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells or spores may be absent, or present at, for example, a concentration of at least 1×10⁴, 1×10⁵, 1×10⁶, 1×10⁷, 1×10⁸, 1×10⁹, 1×10¹⁰, 1×10¹¹ or 1×10¹² or more CFU per milliliter of the composition.

As used herein, a “microbe-based product,” is a product to be applied in practice to achieve a desired result. The microbe-based product can be simply a microbe-based composition harvested from the cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “reduces” means a negative alteration of at least 1%, 5%, 10%, 25%, 50%, 75%, or 100%.

As used herein, “surfactant” means a compound that lowers the surface tension (or interfacial tension) between two liquids, between a liquid and a gas, or between a liquid and a solid. A “biosurfactant” is a surface-active substance produced by a living cell.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially” of limits the scope of a claim to the specified materials or steps “and those that do not materially affect the basic and novel characteristic(s)” of the claimed invention. Use of the term “comprising” contemplates other embodiments that “consist” or “consist essentially” of the recited components(s).

Unless specifically stated or obvious from context, as used herein, the term “or” is understood to be inclusive. Unless specifically stated or obvious from context, as used herein, the terms “a,” “and,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods of Co-Cultivation

The subject invention provides materials and methods for the production of biomass (e.g., viable or inactive cellular material), extracellular metabolites, and/or intracellular components. In preferred embodiments, the subject invention provides improved methods for producing one or more microbial growth by-products, wherein the methods comprise co-cultivating two or more different microorganisms in a fermentation reactor.

Advantageously, the total cell biomass and/or the total production of the one or more growth by-products achieved when using the subject co-cultivation methods can be greater compared to when cultures of the individual microbes are cultivated separately.

More specifically, in preferred embodiments, the subject invention provides methods for enhanced production of one or more microbial growth by-products, the methods comprising co-cultivating a first microorganism and a second microorganism in a submerged fermentation reactor under conditions favorable for growth and production of the one or more growth by-products. In certain embodiments, the first microorganism is a myxobacterium and the second microorganism is a strain of Pseudomonas.

The microorganisms can be co-cultivated using cultivation systems ranging from small to large scale. These cultivation systems can include, but are not limited to, submerged cultivation/fermentation, solid state fermentation (SSF), and hybrids, modifications and/or combinations thereof.

In certain preferred embodiments, the methods for co-cultivating microorganisms and/or for producing microbial growth by-products comprise: inoculating a fermentation system comprising a liquid nutrient medium with a first microorganism and inoculating the fermentation system with a second microorganism, wherein the first microorganism is a Myxococcus spp. bacterium and the second microorganism is a strain of Pseudomonas spp. Even more preferably, in one embodiment, the Myxococcus is M. xanthus and the Pseudomonas is a strain of P. chlororaphis. Preferably, the strain is not pathogenic to humans and/or animals.

In one embodiment, the strain of P. chlororaphis is P. chlororaphis subsp. aureofaciens “strain 306” (Ukrainian Collection of Microorganisms (UCM) deposit number UCM B-306 (IMV B-7096), no. 2687).

In one embodiment, the strain of P. chlororaphis is P. chlororaphis no. 2116).

In certain embodiments, the co-cultivation method utilizes submerged fermentation. In certain embodiments, a hybrid of solid state and submerged fermentation is used, wherein a particulate anchoring carrier is suspended in the liquid culture medium to serve as a site for cell attachment and/or biofilm formation. This is particularly useful for the growth of myxobacteria, which can exhibit enhanced growth on a solid surface or other carrier.

The microbe growth vessel used according to the subject invention can be any fermenter or cultivation reactor for industrial use. In one embodiment, the vessel may have functional controls/sensors or may be connected to functional controls/sensors to measure important factors in the co-cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken at any point throughout fermentation in order to perform, e.g., CFU count and/or purity measurements. In one embodiment, sampling is performed at the start of fermentation, and multiple times per day (e.g., twice per day) throughout fermentation.

In some embodiments, the cultivation method utilizes fed-batch cultivation. The fermentation reactor can be fed with, for example, canola oil (or other anti-foam/de-foaming solution), carbon sources (e.g., glycerol), pH adjusters, and/or other additional nutrient sources as needed.

In one embodiment, the fermentation reactor is connected to a feed container. The feed container preferably holds liquid nutrient medium and/or the other substances for feeding (e.g., transferring or supplementing), into the fermentation reactor. “Feeding” of the fermentation reactor can occur either continuously or at designated time points throughout cultivation.

In certain embodiments, the designated feed time points are at about 12 hours, 24 hours, 36 hours, 48 hours or 52 hours after the start of cultivation. In certain embodiments, there are multiple time points at which the nutrient medium and/or other feed substances are fed into the reactor, for example, every 6 hours, every 12 hours, every 24 hours, every 36 hours, or every 48 hours throughout cultivation.

In one embodiment, the fermentation reactor is connected to a foam collection container. Despite the use of an anti-foam/de-foaming solution in the nutrient medium and/or feed, some amounts of foam are still naturally produced by the fermentation process. In some embodiments, foam is automatically and/or manually extracted from the reactor and collected in the foam collection container. In some embodiments, the collected foam comprises microbial growth by-products, such as biosurfactants, that can be extracted and optionally, purified.

In one embodiment, the liquid nutrient medium comprises a carbon source. The carbon source cam be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, rice bran oil, olive oil, corn oil, sesame oil, canola oil and/or linseed oil; powdered molasses, etc. These carbon sources may be used independently or in a combination of two or more.

In one embodiment, the liquid nutrient medium comprises a nitrogen source. The nitrogen source can be, for example, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid nutrient medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron (ferrous) sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, peptone, yeast extract, potato extract, beef extract, soybean extract, banana peel extract, and the like, or in purified forms. Amino acids such as, for example, those useful for biosynthesis of proteins, can also be included.

In some embodiments, the particulate anchoring carrier is suspended in the liquid culture medium prior to, concurrently with, or after the liquid culture medium is inoculated with the first and/or second microorganisms.

The particulate anchoring carrier can be any material suitable for serving as a nucleation site for bacterial attachment and growth. In some embodiments, the material comprises a plurality of individual pieces, particles, and/or grains, which are about 0.1 μm to about: 5 mm, 4 mm, 3 mm, 2 mm, 1 mm or 0.5 mm in diameter. Bacteria will attach to the pieces and accumulate thereon, producing bacterial-carrier masses.

The anchoring carrier can be inert, or it can carry and/or comprise additional nutrients and/or microbial inoculant. In certain embodiments, the anchoring carrier can be porous. The anchoring carrier can comprise synthetic materials and/or naturally-derived materials.

In one embodiment, the anchoring carrier comprises sodium alginate beads. The beads can be prepared by, for example, continuously adding a solution comprising 1 to 5%, or 2 to 3% aseptic sodium alginate and, optionally, nutrients and/or bacterial inoculant, into a sterile 1 to 7%, or 2 to 5% calcium chloride solution to form beads.

In one embodiment, the anchoring carrier can comprise balls made of, for example, glass, a polymer (e.g., polylactic acid (PLA)), agar, or gelatin. In one embodiment, the anchoring carrier can be pieces of, for example, a chopped sponge or loofa. In one embodiment, the anchoring carrier can comprise foodstuff, for example, seeds, nuts, beans or even pieces of chopped fruit, such as bananas.

In preferred embodiments, the anchoring carrier comprises fine grains of cellulose and/or corn flour. In one embodiment, the use of fine grains is preferred over larger particles (e.g., greater than 5 mm), because it facilitates scaling-up of the process.

Advantageously, the use of the anchoring carrier provides for increased production of bacterial biomass due to, for example, the increased surface area to which the bacteria can attach and accumulate. Additionally, the accumulation of bacterial biomass can lead to increases in the production of beneficial growth by-products, such as biosurfactants.

In one embodiment, bacteria grow in the form of a biofilm on the anchoring carrier. In one embodiment, some bacteria grow in the liquid culture medium in planktonic form, and some bacteria grow on the anchoring carrier.

In some embodiments, the liquid culture medium is inoculated with the microorganisms prior to, or concurrently with, suspension of the anchoring carrier. In some embodiments, the anchoring carrier is pre-inoculated with the first and/or second microorganism before being suspended in the liquid culture medium. The method of co-cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid. In certain embodiments, dissolved oxygen (DO) levels are maintained at about 25% to about 75%, about 30% to about 70%, about 35% to about 65%, about 40% to about 60%, or about 50% of air saturation. Air flow can be supplied at, for example, about 0.5 to about 2.0 v/m, or about 1.0 to about 1.5 vvm.

In some embodiments, the method for co-cultivation may further comprise adding acids and/or antimicrobials in the liquid medium before and/or during the co-cultivation process to protect from contamination.

In one embodiment, prior to inoculation, the components of the liquid culture medium can optionally be sterilized. If used, the anchoring carrier is also preferably sterilized, for example, using an autoclave or other method known in the art. Additionally, water used for preparing the medium can be filtered to prevent contamination.

In one embodiment, sterilization of the liquid nutrient medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for co-cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the mixture should be suitable for the microorganism of interest. In some embodiments, the pH is about 2.0 to about 11.0, about 3.0 to about 10.0, about 4.0 to about 9.0, about 5.0 to about 8.0, or about 6.0 to about 7.0. In one embodiment, the pH is about 6.8. Buffers, and pH regulators, such as carbonates and phosphates, may be used to stabilize pH near a preferred value. In certain embodiments, a basic solution (e.g., 15 to 25%, or 20% NaOH solution) is included in the liquid nutrient medium and/or is fed into the reactor during cultivation to automatically maintain and/or adjust pH of the culture. When metal ions are present in high concentrations, use of a chelating agent in the liquid medium may be necessary.

In one embodiment, the method for co-cultivation of microorganisms is carried out at about 5° to about 100° C., about 15° to about 60° C., about 20° to about 45° C., or about 24° to about 30 ° C. In one embodiment, the co-cultivation may be carried out continuously at a constant temperature. In another embodiment, the co-cultivation may be subject to changing temperatures.

According to the subject methods, the first and second microorganisms can be incubated in the fermentation system for a time period sufficient to achieve a desired effect, e.g., production of a desired amount of cell biomass or a desired amount of one or more microbial growth by-products. The biomass content may be, for example from 5 g/l to 180 g/1 or more, or from 10 g/1 to 150 g/l.

In some embodiments, fermentation occurs for 24 hours to 1 week, about 4 to 6 days, or about 5 days. The microbial growth by-product(s) produced by microorganisms may be retained in the microorganisms or secreted into the growth medium. In certain embodiments, the growth by-product is produced in the form of a foam layer at the top of the culture.

In another embodiment, the method for producing microbial growth by-products may further comprise steps of extracting, concentrating and/or purifying the microbial growth by-product of interest. Alternatively, the microbial growth by-products can be utilized in their crude form, meaning no purification is performed. In a further embodiment, the growth medium may contain compounds that stabilize the activity of the microbial growth by-product.

The methods can be performed in a batch, quasi-continuous, continuous process, or a fed-batch process.

In one embodiment, all of the foam, nutrient medium, cells and/or bacterial-carrier masses are removed upon the completion of the co-cultivation (e.g., upon, for example, achieving a desired cell density, or amount of metabolite). The remaining cell mass can be recycled and/or hydrolyzed to obtain any leftover compounds present in the cells. In this batch procedure, an entirely new batch is initiated upon harvesting of the first batch.

In one embodiment, the process is a fed-batch process, where certain nutrient sources and/or other substances are fed into the reactor at certain time points to replenish the nutrient medium and/or to increase the efficiency of the process. The entire batch is harvested at the end of the cultivation cycle, and an entirely new batch is initiated upon harvesting of the first batch.

In one embodiment, the process is continuous or quasi-continuous, where the growth by-products of interest are collected from the culture, for example, from the foam that forms during co-cultivation and/or from the liquid nutrient medium. In preferred embodiments, the foam and/or medium is placed into a collection container with an optional pH meter. Biomass and/or inoculated anchoring carriers with viable cells remain in the fermentation reactor as an inoculant and the nutrient medium is replenished, e.g., from a feed tank housing fresh nutrient medium, to continue microbial growth and production of metabolites.

In one embodiment, the foam can be extracted on a consistent basis, meaning every 1 to 24 hours, every other day, or every 2 to 7 days. In another embodiment, the foam can be extracted upon reaching a certain volume. The composition that is removed can be a cell-free foam or broth, and/or it can contain some cells.

Foam and/or broth that are collected from the cultivation system can be processed by washing and/or centrifuging to extract the microbial growth by-products. Optionally, the growth by-products can then be stored, purified, and/or used directly in crude form.

In one embodiment, some or all of the anchoring carriers, if used, can be harvested from the culture and washed using a solvent, for example, low concentration (e.g., 1 to 2%) ethanol. The resulting liquid is then centrifuged to separate growth by-products and cell mass.

Advantageously, the total cell biomass and/or the total production of the one or more growth by-products achieved when using the subject co-cultivation methods can be greater compared to when pure cultures of the individual microbes are cultivated on their own.

In certain embodiments, the total cell biomass achieved according to the subject methods is at least 0.01%, 1%, 5%, 10%, 20%, 30%, 40%, 50%, 60%, 70%, 80%, 90%, 100%, or more, greater than when the first and second microorganisms are cultivated individually.

In certain embodiments, the total concentration of a growth by-product produced according to the subject methods is at least 0.01%, 1%, 5%, 10%, 20%, 30%, 40%, 50%, 60%, 70%, 80%, 90%, 100%, or more, greater than when the first and second microorganisms are cultivated individually.

### Microorganisms

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microorganisms are bacteria, including Gram-positive and Gram-negative bacteria. In specific embodiments, the first microorganism is selected from myxobacteria. Myxobacteria are slime-forming, predatory bacteria that live in groups, or swarms. These swarms may form complex biofilms, as well as fruiting body structures, which are either simple or branched aggregates containing myxospores. During predation, the bacteria secrete predatory molecules, including enzymes, antibiotics and other secondary metabolites, which can include, for example, biosurfactants.

Myxobacteria include, for example, Myxococcus spp., Stignatella aurantiaca, Sorangium cellulosum, Minicystis rosea, and Chondromyces crocatus.

In preferred embodiments, the myxobacteria is a Myxococcus spp. bacterium selected from, for example, M. xanthus, M. fulvus, M. flavescens, M. macrosporus, M. stipitatus, M. virescens, M. coralloides, and M. disciformis. Even more preferably, the Myxococcus is M. xanthus.

In certain embodiments, the second microorganism is selected from Pseudomonas spp. bacteria. Preferably, the Pseudomonas is a strain that is not pathogenic to humans or animals.

The Pseudomonas genus comprises Gram-negative bacteria that typically grow in aerobic conditions. Some are pathogenic to humans (e.g., P. aeruginosa) or plants (e.g., P. putida), while others can promote growth in plants (e.g., P. fluorescens). Most pseudomonads form biofilms, making them particularly resistant to antibiotics and difficult to treat in the case of an infection; however, many Pseudomonas spp. are capable of producing useful growth by-products, including, for example, biosurfactants. In one embodiment, the strain of P. chlororaphis is P. chlororaphis subsp. aureofaciens “strain 306” (Ukrainian Collection of Microorganisms (UCM) deposit number UCM B-306 (IMV B-7096), no. 2687). In one embodiment, the strain of P. chlororaphis is P. chlororaphis subsp. aureofaciens “strain 111” (UCM deposit number UCM B-111 (IMV B-7097), no. 2116).

In certain embodiments, one or more additional microorganisms are included, in addition to the first and second microorganisms. In some embodiments, the additional microorganism(s) is a strain of Pseudomonas other than that which is utilized as the second microorganism. For example, in some embodiments, strain 306 and strain 111 are both co-cultivated with the first microorganism.

In preferred embodiments, M. xanthus and a strain of P. chlororaphis are co-cultivated according to the subject methods. Advantageously, in some embodiments, the cell biomass from co-cultivation of these two strains is greater than when pure cultures of the individual microbes are cultivated. Furthermore, in some embodiments, production of biosurfactants and/or other metabolites in co-culture is greater than when pure cultures of the individual microbes are used.

In certain embodiments, this enhanced production of growth by-products and/or metabolites is caused by the co-cultivation, wherein the presence of a competitor microorganism induces enhanced production of, for example, defensive molecules and/or self-growth promoters. In certain embodiments, these are biosurfactants and/or terpenes/terpenoids.

### Microbial Growth By-Products

The methods and systems of the subject invention can be used to produce compositions comprising one or more useful microbial growth by-products.

In preferred embodiment, the growth by-products are one or more biosurfactants. Biosurfactants according to the subject invention can include, for example, glycolipids, lipopeptides, flavolipids, phospholipids, fatty acid esters, fatty acid ethers, lipoproteins, lipopolysaccharide-protein complexes, and/or polysaccharide-protein-fatty acid complexes.

In specific embodiments, the one or more biosurfactants are rhamnolipids (RLP) or similar glycolipid biosurfactants. Rhamnolipids are comprise a glycosyl head group (i.e., a rhamnose) moiety, and a 3-(hydroxyalkanoyloxy)alkanoic acid (HAA) fatty acid tail, such as, e.g., 3-hydroxydecanoic acid. Two main classes of rhamnolipids exist, mono- and di-rhamnolipids, which comprise one or two rhamnose moieties, respectively. The HAA moiety can vary in length and degree of branching, depending on, for example, the growth medium and the environmental conditions.

In some embodiments, the biosurfactant is a rhamnolipid (RLP) or similar glycolipid biosurfactants. Rhamnolipids comprise a glycosyl head group (i.e., a rhamnose) moiety, and a 3-(hydroxyalkanoyloxy)alkanoic acid (HAA) fatty acid tail, such as, e.g., 3-hydroxydecanoic acid. Two main classes of rhamnolipids exist, mono- and di-rhamnolipids, which comprise one or two rhamnose moieties, respectively. The HAA moiety can vary in length and degree of branching, depending on, for example, the growth medium and the environmental conditions.

In certain embodiments, the RLP has a general structure according to General Structure (1):

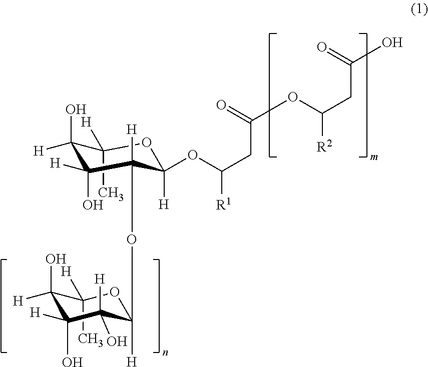

where m is 2, 1 or 0,

n is 1 or 0,

R¹ and R² are, independently of one another, the same or a different organic functional group having 2 to 24, preferably 5 to 13 carbon atoms, in particular a substituted or unsubstituted, branched or unbranched alkyl functional group, which can also be unsaturated,

wherein the alkyl functional group is a linear saturated alkyl functional group having 8 to 12 carbon atoms, or is a nonyl or a decyl functional group or a mixture thereof.

Salts of these compounds are also included according to the invention. In the present invention, the term “di-rhamnolipid” is understood to mean compounds of the above formula or the salts thereof in which n is 1. Accordingly, “mono-rhamnolipid” is understood in the present invention to mean compounds of the general formula or the salts thereof in which n is 0.

In certain embodiments, the methods can be used to produce from about 0.1 to about 30 g/L, about 1 to about 25 g/L, or about 5 to about 25 g/L of RLP and/or RLP-like glycolipids.

In some embodiments, the microorganisms can also produce one or more additional types of biosurfactants, such as other glycolipids (e.g., sophorolipids, trehalose lipids, cellobiose lipids and/or mannosylerythritol lipids) and/or flavolipids. Flavolipids are typically produced by bacteria of the genus Flavobacterium. The hydrophilic moiety comprises citric acid and two cadaverine molecules. The hydrophobic moiety is composed of two branched-chain acyl groups ranging from 6 to 10 carbons.

In certain embodiments, the methods can be used to produce about 0.1 to about 30 g/L about 1 to about 25 g/L, or about 5 to about 25 g/L of the one or more other types of biosurfactants, e.g., flavolipids.

In some embodiments, the microbial growth by-products include other metabolites. As used herein, a “metabolite” refers to any substance produced by metabolism (e.g., a growth by-product), or a substance necessary for taking part in a particular metabolic process, for example, enzymes, enzyme inhibitors, biopolymers, acids, solvents, gases, proteins, peptides, amino acids, alcohols, pigments, polyketides, pheromones, hormones, lipids, ectotoxins, endotoxins, exotoxins, carbohydrates, antibiotics, anti-fungals, anti-virals and/or other organic and/or bioactive compounds. The metabolite content produced by the method can be, for example, at least 0.1%, 1%, 5%, 10%, 20%, 30%, 40%, 50%, 60%, 70%, 80%, or 90%.

In certain embodiments, the one or more growth by-products include organic compounds, such as terpenes and/or terpenoids. Terpenes and terpenoids (isoprenoids) are hydrocarbon compounds typically produced by plants and some insects. Because of the growth rate of terpene/terpenoid-producing plants, such as conifers and cannibus, mass production of terpenes and terpenoids can also be very slow and uneconomical. In some embodiments, the microorganisms cultivated according to the subject invention produce terpenes and/or terpenoids.

Types of terpenes can include, for example, hemiterpenes, monoterpenes, sesquiterpenes, diterpenes, sesterterpenes, triterpenes, sesquarterpenes, tetraterpenes, polyterpenes, and/or norisoprenoids. Terpenoids are modified terpenes. Types of terpenoids can include, for example, hemiterpenoids, monoterpenoids, sesquiterpenoids, diterpenoids, sesterterpenoids, triterpenoids, tetraterpenoids, and/or polyterpenoids. Specific, non-limiting examples of terpenes and/or terpenoids include carotene, carotenoids, myrcene, limonene, linalool, pinene, caryophyllene, bisabolol, citral, menthol, camphor, salvinorin A, cannabinoids, ginkgolide, bilobalide, and curcuminoids. In one embodiment, the organic compound is a carotenoid.

In certain embodiments, the one or more growth by-products include enzymes such as, for example, oxidoreductases, transferases, hydrolases, lyases, isomerases and/or ligases. Specific types and/or subclasses of enzymes according to the subject invention can also include, but are not limited to, nitrogenases, proteases, amylases, glycosidases, cellulases, glucosidases, glucanases, galactosidases, moannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases (e.g., alkane hydroxylase), esterases, lipases, ligninases, mannanases, oxidases, laccases, tyrosinases, cytochrome P450 enzymes, peroxidases (e.g., chloroperoxidase and other haloperoxidases), and lactases.

In certain embodiments, the one or more growth by-products include antibiotic compounds, such as, for example, aminoglycosides, amylocyclicin, bacitracin, bacillaene, bacilysin, bacilysocin, corallopyronin A, difficidin, etnangien gramicidin, β-lactams, licheniformin, macrolactinsublancin, oxydifficidin, plantazolicin, ripostatin, spectinomycin, subtilin, tyrocidine, and/or zwittermicin A. In some embodiments, an antibiotic can also be a type of biosurfactant.

In certain embodiments, the one or more growth by-products include anti-fungal compounds, such as, for example, fengycin, surfactin, haliangicin, mycobacillin, mycosubtilin, and/or bacillomycin. In some embodiments, an anti-fungal can also be a type of biosurfactant.

In certain embodiments, the one or more growth by-products include other bioactive compounds, such as, for example, butanol, ethanol, acetate, ethyl acetate, lactate, acetoin, benzoic acid, 2,3-butanediol, beta-glucan, indole-β-acetic acid (IAA), lovastatin, aurachin, kanosamine, reseoflavin, terpentecin, pentalenolactone, thuringiensin (β-exotoxin), polyketides (PKs), terpenes, terpenoids, phenyl-propanoids, alkaloids, siderophores, as well as ribosomally and non-ribosomally synthesized peptides, to name a few.

### Microbe-based Products

The subject invention provides microbe-based products, as well as their use in a variety of applications, including, for example, agriculture, enhanced oil recovery, bioremediation, pharmaceuticals, and cosmetics.

One microbe-based product of the subject invention is simply the fermentation medium containing the microorganisms, microbial growth by-products produced by the microorganisms, any residual nutrients and/or residual particulate anchoring carrier. The microbe-based product may be used with or without extraction and/or purification.

The microorganisms may be in an active or inactive form, or in the form of vegetative cells, biofilm, spores, or a combination thereof. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

In one embodiment, the first and second microorganisms are separated from each other after co-cultivation. In one embodiment, the product comprises a blend of the first and second microorganisms and/or their growth by-products.

In one embodiment, the composition does not comprise live microorganisms. In one embodiment, the composition does not comprise microorganisms at all, whether live or inactive.

In one embodiment, the composition comprises the one or more microbial growth by-products separated from the microorganism that produced them. The growth by-products can be in a purified or unpurified form.

The microorganisms, nutrient medium and/or foam resulting from the microbial growth can be removed from the fermenter and/or collection container and transferred via, for example, piping for immediate use.

In other embodiments, the composition (microbes, foam and/or broth) can be placed in containers of appropriate size, taking into consideration, for example, the intended use, the contemplated method of application, the size of the fermentation tank, and any mode of transportation from microbe growth facility to the location of use. Thus, the containers into which the microbe-based composition is placed may be, for example, from 1 gallon to 1,000 gallons or more. In certain embodiments the containers are 2 gallons, 5 gallons, 25 gallons, or larger.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100%, 10% to 90%, 20% to 80%, or 30% to 70%, inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, the product is stored at or below a temperature such as, for example, 20° C., 15° C., 10° C., 5° C. or 4° C., or less. If cells are present and in spore form, the product is, in one embodiment, stored and transported at a low temperature, not higher than 15 ° C., in order to prevent premature germination.

### Methods of Use

The microbe-based products of the subject invention can be used for a variety of purposes. In one embodiment, the composition can be used in agriculture. For example, methods are provided wherein a composition produced according to the subject invention is applied to a plant and/or its environment to treat and/or prevent the spread of pests and/or diseases. The composition can also be useful for enhancing water dispersal and absorption in the soil, as well as enhance nutrient absorption from the soil through plant roots, facilitate plant health, increase yields, and manage soil aeration.

In one embodiment, the subject compositions can be highly advantageous in the context of the oil and gas industry. When applied to an oil well, wellbore, subterranean formation, or to equipment used for recovery oil and/or gas, the compositions produced according to the subject invention can be used in methods for enhancement of crude oil recovery; reduction of oil viscosity; removal and dispersal of paraffin from rods, tubing, liners, and pumps; prevention of equipment corrosion; recovery of oil from oil sands and stripper wells; enhancement of fracking operations as fracturing fluids; reduction of H₂S concentration in formations and crude oil; and cleaning of tanks, flowlines and pipelines.

In one embodiment, the compositions produced according to the subject invention can be used to improve one or more properties of oil. For example, methods are provided wherein the composition is applied to oil or to an oil-bearing formation in order to reduce the viscosity of the oil, convert the oil from sour to sweet oil, and/or to upgrade the oil from heavy crude into lighter fractions.

In one embodiment, the compositions produced according to the subject invention can be used to clean industrial equipment. For example, methods are provided wherein a composition is applied to oil production equipment such as an oil well rod, tubing and/or casing, to remove heavy hydrocarbons, paraffins, asphaltenes, scales and other contaminants from the equipment. The composition can also be applied to equipment used in other industries, for example, food processing and preparation, agriculture, paper milling, and others where fats, oils and greases build up and contaminate and/or foul the equipment.

In one embodiment, the compositions produced according to the subject invention can be used to enhance animal health. For example, methods are provided wherein the composition can be applied to animal feed or water, or mixed with the feed or water, and used to prevent the spread of disease in livestock and aquaculture operations, reduce the need for antibiotic use in large quantities, reduce methane, carbon dioxide and/or nitrous oxide emissions, as well as to provide supplemental proteins and other nutrients.

In one embodiment, the compositions produced according to the subject invention can be used to prevent spoilage of food, prolong the consumable life of food, and/or to prevent food-borne illnesses. For example, methods are provided wherein the composition is applied to a food product, such as fresh produce, baked goods, meats, and post-harvest grains, to prevent undesirable microbial growth.

Other uses for the subject compositions include, but are not limited to, biofertilizers, biopesticides, bioleaching, bioremediation of soil and water, pharmaceutical adjuvants (for increasing bioavailability of orally ingested drugs), cosmetic products, control of unwanted microbial growth, and many others.

### Local Production of Microbe-Based Products

In preferred embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The distributed microbe growth facilities can be located at the location where the microbe-based product will be used. For example, the microbe growth facility may be less than 300, 250, 200, 150, 100, 75, 50, 25, 15, 10, 5, 3, or 1 mile from the location of use.

The microbe growth facilities of the subject invention produces fresh, microbe-based compositions, comprising the microbes themselves, microbial metabolites, and/or other components of the broth in which the microbes are grown. If desired, the compositions can have a high density of vegetative cells or propagules, or a mixture of vegetative cells and propagules.

Because the microbe-based product is generated locally, without resort to the microorganism stabilization, preservation, storage and transportation processes of conventional microbial production, a much higher density of bacteria cells and/or propagules can be generated, thereby requiring a smaller volume of the microbe-based product for use in the on-site application or which allows much higher density microbial applications where necessary to achieve the desired efficacy. This allows for a scaled-down bioreactor (e.g., smaller fermentation tank, and smaller supplies of starter material, nutrients, pH control agents), which makes the system efficient. Local generation of the microbe-based product also facilitates the inclusion of the growth broth in the product. The broth can contain agents produced during the fermentation that are particularly well-suited for local use.

Advantageously, the compositions can be tailored for use at a specified location. The microbe growth facilities provide manufacturing versatility by the ability to tailor the microbe-based products to improve synergies with destination geographies and harness the power of naturally-occurring local microorganisms and their metabolic by-products to improve oil production. Local microbes can be identified based on, for example, salt tolerance and ability to grow at high temperatures. Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated broth and metabolites in which the cells are originally grown.

The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Co-Cultiavtion of M. xanthus and Strain 111 For Production of Biosurfactants And Hydrocarbons

Pseudomonas chlororaphis “strain 111” is grown in a small-scale reactor for at least 48 hours to produce a 3.0-5% inoculum. Myxococcus xanthus is grown in a small-scale reactor for at least 4 days to produce a 0.6-1.0% inoculum. The M. xanthus inoculum can be sampled and tested using slide streaking after 3 days to test for purity.

A fermentation reactor is inoculated with the two inocula. The nutrient medium comprises:

| | |
|---|---|
| Glucose | 1 to 5 g/L |
| Casein peptone | 5 to 20 g/L |
| Glycerol | 10 to 30 g/L |
| Yeast extract | 1 to 5 g/L |
| KH₂PO₄ | 1 to 5 g/L |
| Na₂HPO₄ | 1 to 5 g/L |
| NH₄NO₃ | 0.1 to 0.5 g/L |
| MgSO₄•7H₂O | 0.01 to 0.1 g/L |
| FeSO₄ | 0.001 to 0.05 g/L |
| Trace metals | 0.1 to 1.0 g/L |

Additionally, the nutrient medium includes fine grain particulate anchoring carrier comprising cellulose (1.0 to 5.0 g/L) and/or corn flour (1.0 to 8.0 g/L).

An aqueous base solution comprising 20% NaOH is fed into the reactor to adjust and maintain pH automatically to/at about 6.8 to 7.0. Then, canola and/or vegetable oil (10 to 30 ml/L) is added to reduce foam production in the reactor and serve as an additional source of nutrients. Additional canola/vegetable oil can be fed throughout fermentation as needed to reduce foam and/or supplement the nutrient medium.

Temperature is maintained at about 24° C.; DO is maintained at about 50%; and air flow rate is maintained at about 1 vvm.

Cultivation is carried out for about 5 days. Sampling of the fermenter and the foam collection tank for CFU count and/or purity is performed at 0 hr., then twice per day throughout fermentation. Sampling can also occur at the time that harvesting of the culture occurs, i.e., after 5 days of cultivation.

The bacterial culture comprises biosurfactants and other microbial growth by-products, including orange to red color pigments, indicating the presence of carotenoids (tetraterpenoids).

After the fermentation cycle is finished, the culture is harvested from the reactor. A foam layer comprising microbial growth by-products can also be produced during fermentation. This foam layer is extracted and collected in a collection container. Foam extraction can be conducted throughout fermentation and/or at the end of fermentation.

The harvested culture as well as extracted foam can be processed to purify biosurfactants and organic compounds using, for example, ethyl acetate extraction and/or rotary evaporation purification.

The growth by-products, either in purified or unpurified form, can be analyzed to confirm, for example, the presence of biosurfactants. The growth by-products of strain 111 were measured for surface tension reduction capabilities, indicating the presence of biosurfactants. When added to water, the surface tension was reduced to about 30 mN/m.

### Example 2—Fed-Batch Co-Cultiavtion of M. xanthus And Strain 306 To Produce Biosurfactants

Pseudomonas chlororaphis strain 306 is grown in a small-scale reactor for at least 48 hours to produce a 3.0% inoculum. Myxococcus xanthus is grown in culture flasks (2L working volume) for at least 4 days to produce a 0.5% inoculum. The M. xanthus inoculum can be sampled and tested using slide streaking after 3 days to test for purity.

A fermentation reactor is inoculated with the two inocula. The nutrient medium comprises:

| | |
|---|---|
| Glucose | 1 to 5 g/L |
| Casein peptone | 5 to 20 g/L |
| Glycerol | 10 to 30 g/L |
| Yeast extract | 1 to 5 g/L |
| KH₂PO₄ | 1 to 5 g/L |
| Na₂HPO₄ | 1 to 5 g/L |
| NH₄NO₃ | 0.1 to 0.5 g/L |
| MgSO₄•7H₂O | 0.01 to 0.1 g/L |
| FeSO₄ | 0.001 to 0.05 g/L |
| Trace metals | 0.1 to 1.0 g/L |

Additionally, the nutrient medium includes fine grain particulate anchoring carrier comprising cellulose (1.0 to 5.0 g/L) and/or corn flour (1.0 to 8.0 g/L).

An aqueous base solution comprising 20% NaOH is fed into the reactor to adjust and maintain pH automatically to/at about 6.8 to 7.0. Then, canola and/or vegetable oil (10 to 30 ml/L) is added to reduce foam production in the reactor and serve as an additional source of nutrients.

Cultivation is carried out for about 5 days. Temperature is maintained at about 24° C.; DO is maintained at about 50%; and air flow rate is maintained at about 1 vvm.

Throughout cultivation the reactor is fed with additional canola oil (3%, once every 24 hours) and glycerol (500 g/L, after 48 hours).

Temperature is maintained at about 24° C.; DO is maintained at about 50%; and air flow rate is maintained at about 1 vvm. Sampling of the fermenter and the foam collection tank for CFU count and/or purity is performed at 0 hr., then twice per day throughout fermentation. Sampling can also occur at the time that harvesting of the culture occurs, i.e., after 5 days of cultivation.

After the fermentation cycle is finished, the culture is harvested from the reactor. A foam layer comprising microbial growth by-products can also be produced during fermentation. This foam layer is extracted and collected in a collection container.

The harvested culture, as well as the extracted foam, can be processed to purify biosurfactants using, for example, ethyl acetate extraction and/or rotary evaporation purification.

The growth by-products, either in purified or unpurified form, can be analyzed to confirm, for example, the presence of biosurfactants. The growth by-products of strain 306 were measured for surface tension reduction capabilities, indicating the presence of biosurfactants. When added to water, the surface tension was reduced to about 27 mN/m.

1. A method for enhanced production of one or more microbial growth by-products, the method comprising co-cultivating a first microorganism and a second microorganism in a fermentation reactor,
wherein the first microorganism is a myxobacterium and the second microorganism is a non-pathogenic strain of Pseudomonas, and
wherein a greater concentration of the one or more microbial growth by-products is achieved than would be achieved if the first and second microorganisms were cultivated individually.

2. The method of claim 1, wherein the myxobacterium is a Myxococcus spp. bacterium

3. The method of claim 2, wherein the Myxococcus spp. bacterium is M. xanthus.

4. The method of claim 1, wherein the non-pathogenic strain of Pseudomonas is a strain of P. chlororaphis.

5. The method of claim 4, wherein the strain is P. chlororaphis subsp. aureofaciens strain 306 or P. chlororaphis subsp. aureofaciens strain 111.

6. The method of claim 1, wherein the myxobacterium is M. xanthus and the strain of Pseudomonas is either strain 306 or strain 111.

7. The method of claim 1, wherein the Pseudomonas strain produces the one or more growth by-products.

8. The method of claim 1, wherein the one or more growth by-products are biosurfactants.

9. The method of claim 8, wherein the biosurfactants are rhamnolipids (RLP).

10. The method of claim 8, wherein the biosurfactants are flavolipids.

11. The method of claim 1, wherein the one or more growth by-products are terpenes and/or terpenoids.

12. The method of claim 11, wherein the terpenoids are carotenoids.

13. The method of claim 1, wherein co-cultivating the first and the second microorganisms comprises:
inoculating the fermentation reactor with the first microorganism and inoculating the fermentation reactor with the second microorganism, wherein the fermentation reactor comprises a liquid nutrient medium;
incubating the first and second microorganisms in the reactor under conditions favorable for growth and production of the one or more microbial growth by-products;
extracting the one or more growth by-products from the reactor; and, optionally,
purifying the one or more growth by-products.

14. The method of claim 13, wherein the liquid nutrient medium comprises glucose, casein peptone, glycerol, yeast extract, monopotassium phosphate, disodium phosphate, ammonium nitrate, magnesium sulfate heptahydrate, ferrous sulfate, and trace metals.

15. The method of claim 13, further comprising suspending a particulate anchoring carrier in the liquid nutrient medium.

16. (canceled)

17. The method of claim 15, wherein the first and/or second microorganism attaches to the particulate anchoring carrier and accumulates thereon in the form of a biofilm to form a plurality of bacterial-carrier masses.

18. The method of claim 13, further comprising adding an aqueous base solution comprising 15 to 25% NaOH to the reactor.

19. The method of claim 13, further comprising feeding 500 g/L of glycerol into the reactor after 48 hours of incubation.

20. The method of claim 13, further comprising feeding 3% canola oil into the reactor every 24 hours.

21-23. (canceled)

24. A composition comprising one or more microorganisms and/or one or more microbial growth by-products, said one or more microorganisms comprising Myxococcus xanthus and Pseudomonas chlororaphis, and said one or more microbial growth by-products comprising biosurfactants, terpenes, and/or terpenoids.

25. The composition of claim 24, wherein the biosurfactants are rhamnolipids and/or flavolipids.

26. (canceled)

27. The composition of claim 24, wherein the P. chlororaphis is strain 111 or strain 306.
